SEQUENCE LISTING (3 sequences)

SEQ ID NO: 1 - protein, 30 aa, Artificial Sequence
  Description of Artificial Sequence Synthetic polypeptide
HXEGTFTSDVSSYLEGQAAKEFIAWLVKXR

SEQ ID NO: 2 - protein, 39 aa, Artificial Sequence
  Description of Artificial Sequence Synthetic polypeptide
HGEGTFTSDLSKQMEEEAVRLFIEWLKNGGPSSGAPPPS

SEQ ID NO: 3 - protein, 44 aa, Artificial Sequence
  Description of Artificial Sequence Synthetic polypeptide
HGEGTFTSDLSKQMEEEAVRLFIEWLKNGGPSSGAPPSKKKKKK

(19) United States
(12) Patent Application Publication
(10) Pub. No.: US 2010/0183876 A1
(43) Pub. Date: Jul. 22, 2010

(54) Process for the Preparation of a Peptide Powder Form

(76) Inventors: André Hell, Willer (FR); Michael Jansen, Bartenheim (FR); Michael Rothe, Loerrach (DE); Remy Specker, Fislis (FR); Peter Steidle, Arboldswil (CH); Daniel Strub, Breitenbach (CH); Francis Vix, Riedisheim (FR); Christian Walch, Rixheim (FR)

Correspondence Address: HOFFMANN-LA ROCHE INC.;PATENT LAW DEPARTMENT, 340 KINGSLAND STREET, NUTLEY, NJ, 07110, US

(21) Appl. No.: 12/630,872

(22) Filed: Dec. 4, 2009

(30) Foreign Application Priority Data
Dec. 23, 2008 (EP) 08172689.5

(51) Int. Cl. C07K 14/605; C07K 14/46

(52) U.S. Cl. 428/402; 530/308; 530/324

(57) ABSTRACT

The invention comprises a process for the production of a freely flowable homogenous powder form of a GLP-1 peptide analogue. The process is characterized in that a solution of the peptide analogue in an aqueous organic solvent that is preferably directly obtained from the chromatographic purification process, is subjected to a spray drying process and recovered in the form of a freely flowable homogenous powder.

### PRIORITY TO RELATED APPLICATION(S)

This application claims the benefit of European Patent Application No. 08172689.5, filed Dec. 23, 2008, which is hereby incorporated by reference in its entirety.

### FIELD OF THE INVENTION

The invention refers to the preparation of a peptide powder form, particularly to a freely flowable homogenous powder form of a GLP-1 peptide drug.

All documents cited or relied upon below are expressly incorporated herein by reference.

### BACKGROUND OF THE INVENTION

Suitable peptide drugs are analogues of human glucagon-like peptide-1 (GLP-1), particularly the GLP-1 analogue with the amino acid sequence according to SEQ ID No. 1:

His-Aib-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Aib-Arg-NH₂,

wherein 26 of these amino acids are in the natural L configuration while four are not chiral. Aib means α-aminoisobutyric acid. This peptide is also named (Aib^{8,35})GLP-1(7-36)NH₂ and its pharmaceutical use and preparation by solid phase peptide synthesis (SPPS) are described in the PCT Publication WO 2000/34331.

The synthesis of GLP-1 analogues can also follow a hybrid approach encompassing both solid phase peptide synthesis (SPPS) and fragment couplings in solution. For example the PCT Publication WO 2007/147816 describes the preparation of (Aib^{8,35}) GLP-1(7-36)NH₂ by preparing three fragments and coupling these fragments in solution.

The individual synthetic steps usually are highly selective, however, at the end of a multi-step chemical synthesis the product is typically not pure enough to be used as a drug. The crude product can therefore be subjected to reverse phase high performance liquid chromatography (RP-HPLC), to further purify the peptide and to achieve purity in the range of 96 to 99% (area). After the RP-HPLC stage the product is normally obtained in the form of a solution with a concentration of typically 1 to 15% (w/w) of the peptide.

In order to obtain a dry final product which is suitable for the drug formulation the solution can either be subjected to lyophilization or precipitation techniques.

However, the procedures known in the art suffer from delivering a product in a form which is not freely flowable. Furthermore precipitation and lyophilization techniques are time consuming and need to be applied batchwise.

### SUMMARY OF THE INVENTION

An embodiment provided herein is a process for the production of a freely flowable homogenous powder form of a GLP-1 peptide analogue, wherein a solution of the peptide analogue in an aqueous organic solvent is subjected to a spray drying process and recovered in the form of a freely flowable homogenous powder.

### BRIEF DESCRIPTION OF THE DRAWINGS

The above and other features and advantages of the present invention will become more apparent from the following description of the presently preferred exemplary embodiments of the invention taken in conjunction with the accompanying drawings, in which:

FIG. 1: shows a flow chart of the spray drying process.

FIG. 2a: shows a scanning electron microscopy of a precipitated (Aib^{8,35}) GLP-1(7-36)NH₂

FIG. 2b: shows a scanning electron microscopy of a spray dried (Aib^{8,35}) GLP-1(7-36)NH₂.

### DETAILED DESCRIPTION

The invention provides for a process which is able to deliver a GLP-1 peptide drug in a freely flowable homogenous powder form and which is applicable on technical scale.

The process for the production of a freely flowable homogenous powder form of a GLP-1 peptide analogue is characterized in that a solution of the peptide analogue in an aqueous organic solvent is subjected to a spray drying process and recovered in the form of a freely flowable homogenous powder. The solution of the peptide is directly obtained from the RP-HPLC stage or from a RP-LPLC (low-pressure liquid chromatography) or RP-MPLC (medium-pressure liquid chromatography).

The term “freely flowable” describes the property of spray dried GLP-1 peptide analogues to show favorable flow properties, i.e. the GLP-1 peptide is in a homogenous powder form with no tendency to form aggregates or lumps.

The term “GLP-1 peptide analogue” encompasses the natural human glucagon-like peptide-1 (GLP-1) analogues GLP-1 (7-37) and GLP-1 (7-36)NH₂ and synthetic analogues of the GLP-1 peptide (GLP-1 analogues).

Preferred GLP-1 analogues are the human GLP-1 analogue with the amino acid sequence according to SEQ ID No. 1:

His-Aib-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Aib-Arg-NH₂,

i.e. (Aib^{8,35}) GLP-1(7-36)NH₂, and further analogues as described in the PCT Publication WO 2000/34331. (Aib^{8,35}) GLP-1(7-36)NH₂ is most preferred. The short form designates an analogue formally derived from natural human GLP-1 (1-37) by deleting the amino acid residues Nos. 1 to 6, amidating at the C-terminus and substituting the naturally occurring amino acid residues in position 8 (Ala) and 35 (Gly) by α-aminoisobutyric acid (Aib).

Suitable analogues of the GLP-1 peptide can further be selected from GLP-1 (7-37), GLP-1 (7-36)NH₂, (Gly⁸) GLP-1(7-37), (Gly⁸) GLP-1(7-36), (Ser³⁴)GLP-1 (7-37), (Val⁸)GLP-1 (7-37), (Val⁸,Glu²²) GLP-1 (7-37), (N-ε-(γ-Glu(N-α-hexadecanoyl)))-Lys²⁶Arg³⁴-GLP-1(7-37) (Liraglutide) and D-Ala⁸Lys³⁷-(2-(2-(2-maleimidopropionamido(ethoxy)ethoxy)acetamide)) GLP-1 (7-37) (CJC-1131).

Still further analogues of the GLP-1 peptide can be the exendin analogues selected from exendin-3, exendin-4 (exenatide) having the amino acid sequence according to SEQ ID No. 2:

His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-NH₂,

exendin-4 acid, exendin-4 (1-30), exendin-4 (1-30) amide, exendin-4 (1-28), exendin-4 (1-28) amide, ¹⁴Leu,²⁵Phe exendin-4 amide and ¹⁴Leu,²⁵Phe exendin-4 (1-28) amide as well as AVE-0010, an exendin analogue having the amino acid sequence according to SEQ ID No. 3:

His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-Lys-Lys-Lys-Lys-Lys-Lys-NH₂.

In a preferred embodiment of the invention the process is characterized in that the solution of the peptide analogue in an aqueous organic solvent is directly obtained from preparative HPLC and fed into the spray drying process.

The spray drying process comprises the steps of

a) feeding a solution of the peptide in an aqueous organic solvent from a feed tank (1) through a filter (2);
b) atomizing the filtered solution in a spray chamber (3) with the help of an atomizer (4);
c) mixing the atomized mixture with hot drying gas fed through inlet (5), thereby causing the solvent to evaporate and the peptide powder to precipitate; and
d) feeding the gas powder mixture into a cyclone (6) where the peptide can be collected as freely flowable homogenous powder.

### Step a)

Step a) requires feeding a solution of the peptide in an aqueous organic solvent from a feed tank (1) through a filter (2).

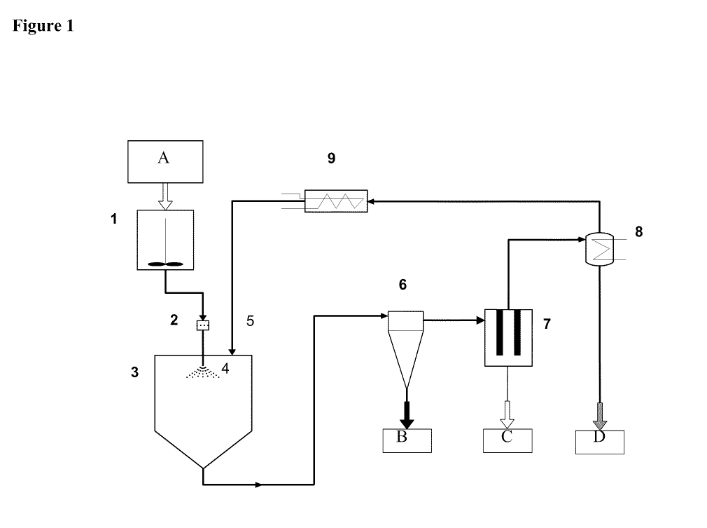

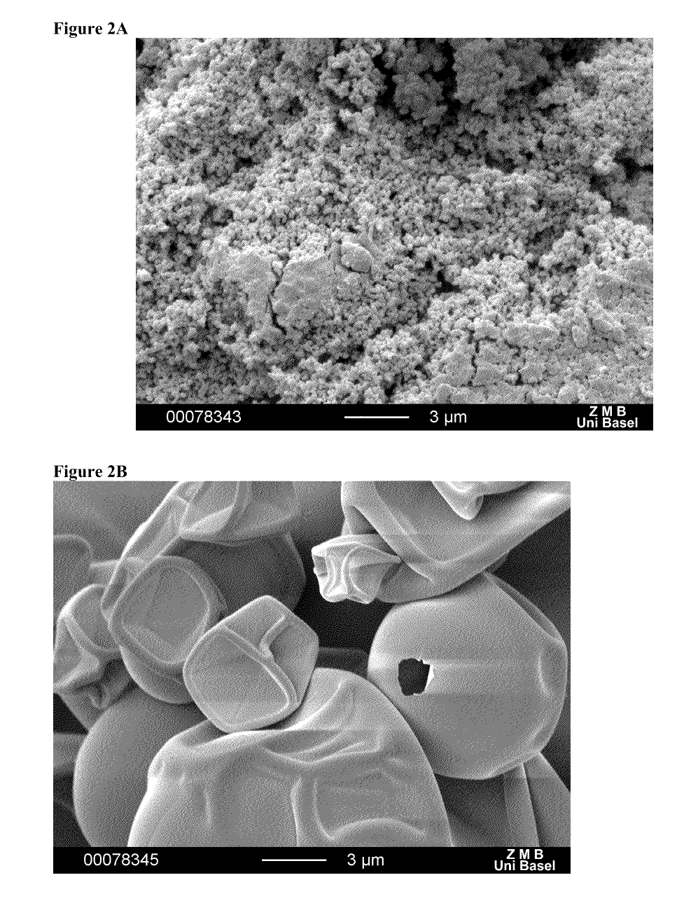

As outlined above the solution of the peptide analogue in an aqueous organic solvent is preferably directly taken from the preparative HPLC (symbolized as “A” in FIG. 1). In case mixtures of water with acetonitrile and THF are used as solvent in the purification step a subsequent concentration of the peptide solution has to be carried out by HPLC or, alternatively, by LPLC (low pressure liquid chromatography) or MPLC (medium pressure liquid chromatography) using a mixture of water with an aliphatic alcohol as solvent.

The aqueous organic solvent usually is a mixture of 20% to 80% w/w of water with 20% to 80% w/w of an aliphatic alcohol, preferably a mixture of 40% to 70% w/w of water with 30% to 60% w/w of an aliphatic alcohol.

The aliphatic alcohol can be selected from methanol, ethanol, n-propanol, 2-propanol, n-butanol, s-butanol or t-butanol, preferably methanol or ethanol.

The peptide content in the aqueous organic solvent as a rule ranges between 0.5% to 15% w/w, preferably between 1.0% to 10% w/w, more preferably between 0.5% to 8% w/w.

The solution of the peptide expediently contains a common buffer for pH stabilization. A suitable buffer is preferably selected from ammonium acetate, which can be dosed in the range of 0.05% to 0.25% w/w, or acetic acid, which can be dosed in the range of 0.05% to 1% w/w. More preferably, the buffer is acetic acid in an amount of 0.05 to 1% w/w.

The solution of the peptide is fed from a feed tank (1) through a filter (2) usually with a feed rate of 1 kg/h to 20 kg/h, preferably of 5 kg/h to 15 kg/h into the spray chamber (3). Depending on the size of the spray-dryer equipment used the feed rate can be consequently increased by a scale-factor of 5 to 10.

The temperature of the solution of the peptide can be selected between 5° C. to 35° C. The size of the filter (2) as a rule is in the range of 0.2 μm to 4.0 μm.

### Step b)

Step b) requires atomizing the filtered solution in a spray chamber (3) with the help of an atomizer (4).

As a rule a rotary wheel atomizer is used. The atomizer speed is selected in a range of 10,000 rpm to 30,000 rpm.

### Step c)

Step c) requires mixing the atomized mixture with hot drying gas feed through inlet (5), thereby causing the solvent to evaporate and the peptide powder to precipitate.

As “hot drying gas” nitrogen, carbon dioxide or air can be used. Preferred “hot drying gas” is nitrogen which can be applied at a temperature of 100° C. to 200° C., preferably 110° C. to 140° C.

The hot drying gas is fed into the spray chamber (3) with a feed rate of 300 kg/h to 500 kg/h. Depending on the size of the spray-dryer equipment used the feed rate can be consequently increased by a scale-factor of 5 to 10.

### Step d)

Step d) requires feeding the gas powder mixture into a cyclone (6) where the peptide can be collected as freely flowable homogenous powder.

The gas fed into the cyclone (6) as a rule has a temperature of 50° C. to 150° C., preferably 50° C. to 110° C. and more preferably 60° C. to 80° C.

The peptide can be collected with equipment well known in the art such as in a bag housing (B).

### Gas Recovery

In a preferred embodiment of the invention the gas can be recovered according to the following gas recovery steps:

e) purifying the gas leaving the cyclone (6) with a filter (7);
f) condensing the aqueous organic solvent in a condenser (8);
g) heating the gas leaving the condenser (8) in a heater (9) and reintroducing the hot drying gas in the spray chamber (3).

Filter (7) is expediently connected to suitable equipment (C) for collecting fine particles withheld in the filter.

Condenser (8) is also connected to suitable equipment (D) for recovering the condensed solvent.

In the heater (9) the gas is again brought to the temperature for use as “hot drying gas” in the spray drying process.

Alternatively, only fresh gas can be used. The spray drying process is then operating in an “open cycle” mode versus operating in a “closed cycle” mode when the gas is recovered.

### Product Characteristics:

The peptide obtained from the spray drying process according to the present invention is as rule in a freely flowable homogenous powder form. The particles accordingly, when compared to a precipitated product, show a lower specific surface area and a lower bulk density. The majority of the particles of the spray dried product are much lower in diameter when compared to the precipitated product.

In a preferred embodiment the freely flowable homogenous powder of (Aib^{8,35})hGLP-1(₇₋₃₆) NH₂ is characterized by a specific surface area measured according to the BET method (ISO 9277) of 0.5 m²/g to 5 m²/g, preferably 0.5 m²/g to 2.5 m²/g i.e. values which are substantially lower than the values measured for the precipitated product.

The spray dried (Aib^{8,35})hGLP-1(₇₋₃₆)NH₂ is further characterized by a particle size distribution d₉₀ (measured by laser scattering) of less than 200 μm, preferably less than 150 μm and more preferably less than 100 μm, which means that 90% of the particles have a particle size of less than 200 μm, preferably less than 100 μm. More than 60% of the precipitated peptide have a particle size exceeding 500 μm.

The mean size of the particles of the spray dried (Aib^{8,35})hGLP-1(₇₋₃₆)NH₂ as a rule is within a range of 10 to 60 μm, preferably within the range of 20 to 40 μm and more preferably within the range of 20 to 30 μm.

The processability parameter bulk density of the spray dried (Aib^{8,35})hGLP-1(₇₋₃₆)NH₂ is as a rule less than 0.25 g/cm³, preferably less than 0.2 g/cm³, but can be adjusted to less than 0.1 g/cm³ and thus is superior when compared to a precipitated product which shows values exceeding 0.3 to 0.4 g/cm³.

It has been found that the bulk density and tapped density of the spray dried product can be adjusted depending on parameters such as the ratio of water to aliphatic alcohol in the aqueous organic solvent (feed solution), the concentration of the peptide and of the acetate in the feed solution and the pH value of the feed solution. With small amounts of acetate in the feed solution bulk densities of the spray dried (Aib^{8,35})hGLP-1(₇₋₃₆)NH₂ of less than 0.1 g/cm³ are obtained, whereas higher amounts of acetate in the feed solution lead to bulk densities of about 0.2 to 0.25 g/cm³.

Thus, a spray dried powder of (Aib^{8,35})hGLP-1(₇₋₃₆)NH₂ with a bulk density of less than 0.1 g/cm³ is obtained by the process as described herein that is characterized in that the solution of the peptide (feed solution) contains less than 2% w/w of (Aib^{8,35})hGLP-1(₇₋₃₆)NH₂ and less than 0.15, preferably less than 0.10% w/w of acetate. A spray dried powder of (Aib^{8,35})hGLP-1(₇₋₃₆)NH₂ with a bulk density of about 0.20 to 0.25 g/cm³ is obtained by the process as described herein that is characterized in that the solution of the peptide (feed solution) contains 7 to 8% w/w of (Aib^{8,35})hGLP-1(₇₋₃₆)NH₂ and acetate in the range of 0.4 to 0.6% w/w.

The following examples shall illustrate the invention without limiting it.

### EXAMPLES

### Example A

### Preparation of The Peptide

The crude peptide (Aib^{8,35})GLP-1(7-36)NH₂ can be prepared according to the method described in WO 2007/147816 by producing three fragments and coupling these fragments in solution.

### Example B

### RP-HPLC Purification

Purification of the crude peptide is performed on a RP (reversed phase) stationary phase. Thus, the sorbent is RP material such as silica gel (e.g. Kromasil 100-16-C18) or acrylic ester macroreticular adsorbent (e.g. Amberchrom CG71M). The purification involves a 1^{st} pass chromatographic purification at a pH of approximately 2, followed by a 2^{nd} pass at a pH of approximately 9.

### 1^{st} Chromatography:

Crude (Aib^{8,35})GLP-1(7-36)NH₂ is dissolved in water/acetonitrile/acetic acid (e.g. 90/9/1 v/v/v) and loaded onto a HPLC column (loading up to 20 g/L, bed depth approx. 25 cm) and the purification program (example for a column with a length of 45 cm is described in Table 1) is initiated. Fractions are collected and may be diluted with water or diluted ammonium hydroxide solution.

TABLE 1
Parameters and Purification Program of Chromatography 1

| Parameter | Description |
|---|---|
| Sorbent | RP silica gel (Kromasil 100-16-C18) |
| Detection | UV |
| Eluent A | aqueous ammonium phosphate (approx. pH 2)/ |
| | acetonitrile (80/20 v/v) |
| Eluent B | aqueous ammonium phosphate (approx. pH 2)/ |
| | acetonitrile (60/40 v/v) |
| Column Flush | aqueous acetic acid or water/acetonitrile (25/75 v/v) |
| Program | |

| | % organic | | |
|---|---|---|---|
| | solvent | Flow | |
| Time (min) | (by weight) | (L/min) | Description |
| 7 | 20 | 5 to 7 | Column equilibration prior to |
| | | | injection |
| initial | 20 | 5 to 7 | Initial conditions after material is |
| | | | loaded on column |
| 1 | 20-30 | 5 to 7 | Initial gradient |
| 40 | 30-35 | 5 to 7 | Second gradient (product elutes) |
| 4 | 70 | 5 to 7 | Column flush |

Proportions of A and B may be varied in order to achieve approximately the % organic solvent indicated in the purification program, corresponding to a minimal retention for the main peak (peptide (Aib^{8,35})GLP-1(7-36)NH₂). The event time, gradient and loading aspects may be varied in order to optimize the purification. The pooled fractions are further purified by the conditions of 2^{nd} Chromatography.

### 2^{nd} Chromatography:

The pooled diluted fractions from Chromatography 1 of (Aib^{8,35})GLP-1(7-36)NH₂ are loaded onto the HPLC column and the purification program (see examples for a 45 cm column in Table 2 and Table 3) is initiated. Fractions are collected and may be diluted with water or diluted acetic acid.

TABLE 2
Parameters and Purification Program of Chromatography 2
(Alternative 2a)

| Parameter | Description |
|---|---|
| Sorbent | RP silica gel (Kromasil 100-16-C18) |
| Detection | UV |
| Eluent C | Aqueous ammonium acetate (approx. pH 9)/ethanol |
| | (80/20 v/v) |
| Eluent D | Aqueous ammonium acetate (approx. pH 9)/ethanol |
| | (35/65 v/v) |
| Column Flush | Aqueous acetic acid/ethanol (10/90 v/v) |
| Program | |

| | % organic | | |
|---|---|---|---|
| | solvent | Flow | |
| Time (min) | (by weight) | (L/min) | Description |
| initial | 15 | 5 to 7 | Initial conditions |
| 1 | 15-35 | 5 to 7 | Initial gradient |
| 30 | 35-44 | 5 to 7 | Second gradient (product elutes) |

Proportions of C and D may be varied in order to achieve approximately the % organic solvent indicated in the purification program, corresponding to a minimal retention for the main peak (peptide (Aib^{8,35}) GLP-1(7-36)NH₂). The event time, gradient and loading aspects may be varied in order to optimize the purification.

The pooled fractions can be directly used in the precipitation process or the spray-drying process as described herein after.

TABLE 3
Parameters and Purification Program of Chromatography 2
(Alternative 2b)

| Parameter | Description | | |
|---|---|---|---|
| Sorbent | RP silica gel (Kromasil 100-10-C4) | | |
| Detection | UV | | |
| Eluent E | Aqueous ammonium acetate (approx. pH 7)/acetonitrile | | |
| | (80/20 v/v) | | |
| Eluent F | Aqueous ammonium acetate (approx. pH 9)/acetonitrile/ | | |
| | THF (25/60/15 v/v/v) | | |
| Column Flush | Aqueous acetic acid/acetonitrile (10/90 v/v) | | |
| Program | | | |
| | % organic | | |
| | solvent | Flow | |
| Time (min) | (by weight) | (L/min) | Description |
| 10 | 12 | 5 to 7 | Column equilibration prior to |
| | | | injection |
| initial | 12 | 5 to 7 | Initial conditions |
| 10 | 12-28 | 5 to 7 | Initial gradient |
| 25 | 28 | 5 to 7 | Isocratic hold |
| 10 | 28-72 | 5 to 7 | Column flush |

Proportions of E and F are varied in order to achieve approximately the % organic solvent indicated in the purification program, corresponding to a minimal retention for the main peak (peptide (Aib^{8,35}) GLP-1(7-36)NH₂). The event time, gradient and loading aspects may be varied in order to optimize the purification.

The pooled fractions are collected and may be diluted with water or diluted acetic acid. They are then loaded directly onto the HPLC column for the following concentration step.

### Concentration of (Aib^{8,35})GLP-1(7-36)NH₂:

The following step for concentration is optionally performed in case alternative 2a is used in the 2^{nd} chromatography step. The pooled, diluted fractions from Chromatography 2 are loaded onto the column and equilibrated with a weak mobile phase (Initial Buffer in Table 4 or aqueous acetic acid/ethanol (85/15 v/v)). The buffer composition is changed to a strong mobile phase (Final Buffer in Table 4 or aqueous acetic acid/ethanol (20/80 v/v)) and (Aib^{8,35})GLP-1(7-36)NH₂ is collected as it elutes from the column.

TABLE 4
Concentration

| Parameter | Description |
|---|---|
| Sorbent | RP material (Kromasil 100-16-C18) |
| Detection | UV |
| Initial Buffer | Aqueous ammonium acetate (approx. pH 9) |
| | ethanol¹ (85/15 v/v) |
| Final Buffer | Aqueous ammonium acetate (approx. pH 9)/ |
| Program | ethanol¹ (20/80 v/v) |

| | % organic | | |
|---|---|---|---|
| | solvent | Flow | |
| Time (min) | (by weight) | (L/min) | Description |
| initial | 28 | 2 to 4 | Initial conditions |
| 1 | 28-35 | 2 to 4 | Initial gradient |
| 20 | 35-70 | 2 to 4 | Second gradient (product elutes) |

¹Ethanol may be substituted by methanol.

The event time, gradient and loading aspects of the column may be varied in order to optimize the concentration. Several concentration runs are combined and the ethanol content of the (Aib^{8,35})GLP-1(7-36)NH₂ containing solution is determined (e.g. by GC).

### Comparative Example C

### Precipitation

In a suitable reactor 184 kg of methyl tert-butyl ether (MTBE) are mixed with 122 kg ethanol at a temperature of 24 to 26° C. 36.7 kg of the purified solution of (Aib^{8,35})hGLP-1(7-36)NH₂ obtained from the preparative HPLC are added within 5 to 15 min. The mixture is heated to a temperature of 34° C. to 36° C., stirred for 1 hour and then cooled to 24° C. to 26° C. After filtration on a filter dryer (0.2 m²) the cake is dried with nitrogen for 15 min and further dried under vacuum (less than 100 mbar) for 9 hours at 25° C. to 30° C. The cake is then washed twice with 3.4 kg ethanol each and dried under vacuum (less than 100 mbar) for 19 hours at 25° C. The obtained product is humidified by passing through the filter dryer from below damp nitrogen for 3 hours and dry nitrogen for 1 hour in an alternating manner until the ethanol content has been depleted to less than 1%. Thereby the product takes up moisture and another drying cycle is necessary. By passing through the filter dryer from below dry nitrogen for 3 hours the moisture content is adjusted to approximately 6%. 601 g of product (Yield: 92% according to HPLC assay) are obtained (containing 97.9% (area) (Aib^{8,35})hGLP-1(7-36)NH₂, 6.1% H₂O, 0.73% ethanol and 0.02% MTBE).

### Spray Drying Examples 1a and 1b

Spray drying conditions applied for example 1a and 1b:

TABLE 5

| | |
|---|---|
| Feed solution composition: | 0.8-1.7% (w/w) of (Aib^{8,35})hGLP-1(₇₋₃₆)NH₂ |
| | 58-79% (w/w) water |
| | 20-40% (w/w) ethanol′ |
| | 0.055-0.11% (w/w) ammonium acetate |
| Feed solution quality: | 97.9 (Aib^{8,35})hGLP-1(₇₋₃₆)NH₂ (area %) |
| Feed Rate | 9-11 kg/h |
| Atomizing Mode | Rotary wheel atomizer |
| Atomizer speed: | 15′000 RPM |
| Drying gas inlet | 135° C. |
| temperature | |
| Drying gas outlet | 76° C. |
| temperature | |
| Drying gas (nitrogen) | 380-420 kg/h, closed cycle mode |
| In-situ” nitrogen purge” | no |

Spray-dried (Aib^{8,35})hGLP-1(₇₋₃₆)NH₂ is of good quality without any new impurities related to thermal degradation (see following Table 6). It contains approximately 1-2% (w/w) of ethanol, 4-5% (w/w) of water as well as 3% (w/w) of acetate (table 4).

TABLE 6
Typical quality attributes of spray-dried (Aib^{8,35})hGLP-1(₇₋₃₆)NH₂

| | Spray-dried | |
|---|---|---|
| | (Aib^{8,35})hGLP-1(₇₋₃₆)NH₂ | |
| Quality Attribute | Example 1a | Example 1b |
| Isolated Amounts | 0.44 kg | 0.15 kg |
| Assay | | |
| (Aib^{8,35})hGLP-1(₇₋₃₆)NH₂ (area %) | 98.0% | 97.6% |
| (Aib^{8,35})hGLP-1(₇₋₃₆)NH₂ (mass %) | 87.6% | 86.9% |
| Organic impurity (mass %) | | |
| RRT = 0.98 | 0.39% | 0.39% |
| RRT = 1.06 | 0.23% | 0.20% |
| RRT = 1.08 | 0.25% | 0.27% |
| Unspecified impurities | 0.77% | 0.92% |
| Total of all organic impurities | 1.6% | 1.8% |
| Residual solvents (% (w/w)) | | |
| Ethanol | 1.1% | 2.0% |
| other organic solvents | <0.01% each | <0.01% each |
| Water % (w/w)) | 4.8% | 4.7% |
| Acetate (% (w/w)) | 2.9% | 3.1% |
| Trifluoroacetate (% (w/w)) | <0.05% | <0.05% |
| Triethylamine (% (w/w)) | 0.04% | 0.04% |
| Ammonium (% (w/w)) | <0.02% | <0.02% |
| Phosphate (% (w/w)) | 0.02% | 0.02% |

nd = not detected

### Examples 2a to 2f

Further small-scale optimization trials were preformed with some variations of the process parameters in order to minimize the amount of residual ethanol:

TABLE 7

| | |
|---|---|
| Feed solution composition: | 1.7% (w/w) of (Aib^{8,35})hGLP-1(₇₋₃₆)NH₂ |
| | 58% (w/w) water |
| | 40% (w/w) ethanol |
| | 0.15% (w/w) ammonium acetate |
| Atomizing Mode | Rotary wheel atomizer |
| Atomizer speed: | 20′000 RPM |

Therefore a continuous “in-situ” nitrogen purge has been set up and a relative depletion of 23 to 28% ethanol has been successfully achieved (table 8).

TABLE 8
Process parameters and specific quality attributes of small-scale trials

| | Examples | | | | | |
|---|---|---|---|---|---|---|
| | Ex. 2a | Ex. 2b | Ex. 2c | Ex. 2d | Ex. 2e | Ex. 2f |
| inlet temperature (° C.) | 135 | 120 | 120 | 135 | 135 | 135 |
| outlet temperature (° C.) | 65 | 70 | 65 | 65 | 65 | 65 |
| gas flow (kg/h) | 350 | 400 | 400 | 350 | 350 | 350 |
| “In-situ” nitrogen purge | no | no | no | no | yes | yes |
| Residual solvents | | | | | | |
| (% (w/w)) | | | | | | |
| Ethanol | 2.2% | 2.2% | 2.4% | 2.3% | 1.7% | 1.8% |
| others each | <0.01% | <0.01% | <0.01% | <0.01% | <0.01% | <0.01% |
| Water (mass %) | 5.6% | 4.0% | 4.0% | 4.8% | n.a. | 3.4% |
| Acetate (mass %) | 2.7% | 2.2% | 2.3% | 2.3% | n.a. | 2.7% |

### Examples 3a and 3b

Subsequent multi kilogram-scale batches were performed using following optimized parameters:

TABLE 9

| | |
|---|---|
| Feed solution composition: | 1.7-1.9% (w/w) of (Aib^{8,35})hGLP-1(₇₋₃₆)NH₂ |
| | 58% (w/w) water |
| | 40% (w/w) ethanol |
| | 0.15% (w/w) ammonium acetate |
| Feed Rate | 11-12 kg/h |
| Atomizing Mode | Rotary wheel atomizer |
| Atomizer speed: | 20′000 RPM |
| Drying gas inlet | 135° C. |
| temperature | |
| Drying gas outlet | 65° C. |
| temperature | |
| Drying gas (nitrogen) | 350 kg/h, closed cycle mode |
| In-situ” nitrogen purge” | continuous |

The obtained quality attributes of two batches are depicted in table 10:

TABLE 10
Quality attributes of large scale batches

| | Spray-dried | |
|---|---|---|
| | (Aib^{8,35})hGLP-1(₇₋₃₆)NH₂ | |
| Quality Attribute | Example 3a | Example 3b |
| Isolated Amounts | 1.9 kg | 2.7 kg |
| Feed solution quality: | 98.1% | 97.7% |
| (Aib^{8,35})hGLP-1(7-36)NH₂ (area %) | | |
| Assay | | |
| (Aib^{8,35})hGLP-1(7-36)NH₂ (area %) | 98.1% | 97.3% |
| (Aib^{8,35})hGLP-1(7-36)NH₂ (mass %) | 88.6% | 88.8% |
| Residual solvents (% (w/w)) | | |
| Ethanol | 1.7% | 1.6% |
| other organic solvents | <0.01% each | <0.01% each |
| Water (% (w/w)) | 4.5% | 4.0% |
| Acetate (% (w/w)) | 2.7% | 2.5% |
| Trifluoroacetate (% (w/w)) | 0.05% | <0.05% |
| Isolation Capacity | 0.16 kg/h | 0.18 kg/h |

### Example 4

160 kg of a solution containing 1.87% (w/w) (Aib^{8,35})hGLP-1(₇₋₃₆)NH₂, 61% (w/w) water and 37% (w/w) ethanol is fed to a Niro SD-4-R-CC (Spraying chamber ø 1.2×0.75 m, capacity 8 kg H₂O/h). After about 15 hours, 2.73 kg of a fine powder (analytical values see example 3a above) are collected from the cyclone.

TABLE 11
Comparison of solid state properties between precipitated and spray-dried
(Aib^{8,35})hGLP-1(₇₋₃₆)NH₂products.
A range obtained from various measurements is provided.

| | Precipitated product | Spray dried |
|---|---|---|
| | (comparison) | product (invention) |
| Specific surface area (BET) | 14.9-18.8 | 0.8-1.4 |
| according to ISO 9277 (m²/g)/ | | |
| Bulk Density (g/cm³) | 0.34-0.39 | 0.05-0.06 |
| Tapped Density (ISO 3953) | 0.38-0.43 | 0.08-0.11 |
| (g/cm³) | | |
| Particle size distribution, | 63% to 90% of the | d₁₀ = 11-12 |
| particle size measured | particles >500 μm | d₅₀ = 33-38 |
| with laser | d₉₀ = 67-86 | |
| scattering method (μm) | | |

### Example 5

Further optimization trials were performed to determine how the bulk density of the spray-dried powder can be adjusted in order to facilitate the down-stream processing. The results are shown in table 12. Examples 5a to 5f were carried out with different compositions of the feed solution, i.e. variations of the solvent ratio (ethanol to water), the concentration of (Aib^{8,35})hGLP-1(₇₋₃₆)NH₂ and the concentration of acetate in the feed solution resulting in different pH. The variations were integrated in the final preparative HPLC step in order to carry out the spray-drying of the preparative HPLC solution directly and without any further modification.

TABLE 12
Evolution of the bulk density of large scale batches

| | Ex. 5a | Ex. 5b | Ex. 5c | Ex. 5d | Ex. 5e | Ex. 5f |
|---|---|---|---|---|---|---|
| Feed solution composition | | | | | | |
| Water/Ethanol (% w/w) | 59/41 | 62/38 | 61/39 | 57/43 | 62/38 | 58/42 |
| Acetate (% w/w) | 0.09 | 0.09 | 0.09 | 0.49 | 0.56 | 0.53 |
| (Aib^{8,35})hGLP-1(₇₋₃₆)NH₂ (% w/w) | 1.67 | 1.87 | 1.92 | 7.06 | 7.25 | 7.48 |
| pH | 8.5 | 8.4 | 8.6 | 5.2 | 5.2 | 5.4 |
| Bulk density (kg/L) | 0.06 | 0.05 | 0.06 | 0.22 | 0.21 | 0.22 |
| Dissolution time | 2 h55 | 2 h58 | 2 h55 | 1 h51 | 1 h41 | 2 h01 |

It is to be understood that the invention is not limited to the particular embodiments of the invention described above, as variations of the particular embodiments may be made and still fall within the scope of the appended claims.

What is claimed is:

1. A process for the production of a freely flowable homogenous powder form of a GLP-1 peptide analogue, wherein a solution of the peptide analogue in an aqueous organic solvent is subjected to a spray drying process and recovered in the form of a freely flowable homogenous powder.

2. The process according to claim 1, wherein the solution of the peptide in an aqueous organic solvent is obtained from preparative HPLC, LPLC or MPLC.

3. The process according to claim 1, wherein the solution of the peptide in an aqueous organic solvent is obtained from preparative HPLC.

4. The process according to claim 1, wherein the spray drying process comprises the steps of
a) feeding a solution of the peptide in an aqueous organic solvent from a feed tank (1) through a filter (2);
b) atomizing the filtered solution in a spray chamber (3) with the help of an atomizer (4);
c) mixing the atomized mixture with hot drying gas fed through inlet (5), thereby causing the solvent to evaporate and the peptide powder to precipitate; and
d) feeding the gas powder mixture into a cyclone (6) where the peptide can be collected as freely flowable homogenous powder.

5. The process according to claim 1, wherein the aqueous organic solvent is a mixture of 20% to 80% w/w of water with 20% to 80% w/w of an aliphatic alcohol.

6. The process according to claim 5, wherein the aliphatic alcohol is methanol or ethanol.

7. The process according to claim 1, wherein the solution of the peptide contains a buffer.

8. The process according to claim 1, wherein the solution of the peptide contains acetate.

9. The process according to claim 1, wherein the solution of the peptide contains 0.4 to 0.6% w/w acetate.

10. The process according to claim 1, wherein the solution of the peptide contains less than 0.15% w/w acetate.

11. The process according to claim 4, wherein in step a) of the spray drying process the solution of the peptide is fed from feed tank (1) through a filter (2) with a feed rate of 1 kg/h to 20 kg/h.

12. The process according to claim 11, wherein the solution of the peptide has a temperature of 5° C. to 35° C.

13. The process according to claim 4, wherein in step a) of the spray drying process the size of the filter (2) is in the range of 0.2 μm to 4.0 μm.

14. The process according to claim 4, wherein in step b) of the spray drying process a rotary wheel atomizer is used.

15. The process according to claim 14, wherein the atomizer speed is selected in a range of 10,000 rpm to 30,000 rpm.

16. The process according to claim 4, wherein in step c) of the spray drying process nitrogen with a temperature of 100° C. to 200° C. is used as hot drying gas.

17. The process according to claim 4, wherein the hot drying gas is fed with a feed rate of 300 kg/h to 500 kg/h.

18. The process according to claim 4, wherein in step d) of the spray drying process the gas fed into the cyclone (6) has a temperature of 50° C. to 110° C.

19. The process according to claim 4, wherein the spray drying process in addition comprises the gas recovery steps of
e) purifying the gas leaving the cyclone (6) with a filter (7);
f) condensing the aqueous organic solvent in a condenser (8);
g) heating the gas leaving the condenser (8) in a heater (9) and reintroducing the hot drying gas in the spray chamber (3).

20. The process according to claim 1, wherein the GLP-1 peptide analogue is selected from the group consisting of GLP-1 (7-37), GLP-1 (7-36)NH₂, (Gly⁸) GLP-1(7-37), (Gly⁸) GLP-1(7-36), (Ser³⁴)GLP-1 (7-37), (Val⁸)GLP-1 (7-37), (Val⁸,Glu²²) GLP-1 (7-37), (Aib^{8,35})hGLP-1(₇₋₃₆)NH₂, (N-ε-(γ-Glu(N-α-hexadecanoyl)))-Lys²⁶Arg³⁴-GLP-1(7-37), D-Ala⁸Lys³⁷-(2-(2-(2-maleimidopropionamido(ethoxy)ethoxy)acetamide)) GLP-1 (7-37), exendin-3, exendin-4, exendin-4 acid, exendin-4 (1-30), exendin-4 (1-30) amide, exendin-4 (1-28), exendin-4 (1-28) amide, ¹⁴Leu,²⁵Phe exendin-4 amide and ¹⁴Leu,²⁵Phe exendin-4 (1-28) amide and AVE-0010.

21. The process according to claim 1, wherein the GLP-1 peptide analogue is the (Aib^{8,35})hGLP-1(7-36)NH₂.

22. A GLP-1 peptide analogue obtainable by a process according to claim 1.

23. A freely flowable homogenous powder form of (Aib^{8,35})hGLP-1(7-36)NH₂.

24. The freely flowable homogenous powder form of (Aib^{8,35})hGLP-1(7-36)NH₂ according to claim 23, wherein the particles have a specific surface area (BET) of 0.5 m²/g to 5 m²/g.

25. The freely flowable homogenous powder form of (Aib^{8,35})hGLP-1(7-36)NH₂ according to claim 23, wherein 90% of the particles have a diameter of less than 200 μm.
